(19) United States
(12) Patent Application Publication
(10) Pub. No.: US 2006/0111345 A1
(43) Pub. Date: May 25, 2006

(54) Pharmaceutical compositions comprising a selective COX-2 inhibitor and a diuretic

(75) Inventors: Reynold Spector, Colts Neck, NJ (US); Colin Gardner, Concord, MA (US)

Correspondence Address: TRANSFORM PHARMACEUTICALS, INC., 29 HARTWELL AVENUE, LEXINGTON, MA, 02421, US

(73) Assignee: Transform Pharmaceuticals, Inc., Lexington, MA (US)

(21) Appl. No.: 11/285,263

(22) Filed: Nov. 21, 2005

Related U.S. Application Data
(60) Provisional application No. 60/630,312, filed on Nov. 23, 2004.

(51) Int. Cl. A61K 31/549; A61K 31/365; A61K 31/415

(52) U.S. Cl. 514/223.5; 514/406; 514/471

(57) ABSTRACT

In a first aspect, the present invention includes a pharmaceutical composition comprising a COX-2 inhibitor and a diuretic. Such a composition can be used to treat a patient suffering from, for example, osteoarthritis, rheumatoid arthritis, juvenile rheumatoid arthritis, pain, primary dysmenorrhea, migraine, or colorectal polyps. The disclosure provides methods as well as pharmaceutical compositions and formulations useful in the treatment and/or prevention of one or more of several conditions described herein.

### CROSS REFERENCE TO RELATED APPLICATIONS

The present application claims the benefit of priority of U.S. Provisional Application Ser. No. 60/630,312, filed Nov. 23, 2004, the contents of which are incorporated herein by reference in its entirety.

### BACKGROUND OF THE INVENTION

Selective COX-2 inhibitors belong to the general class of non-steroidal anti-inflammatory drugs (NSAIDs). Unlike traditional NSAIDs, COX-2 inhibitors are selective inhibitors of cyclooxygenase-II (COX-2) that cause fewer gastric related side effects when administered to a subject.

COX-2 inhibitory drugs include, but are not limited to, rofecoxib, valdecoxib, parecoxib, deracoxib, celecoxib, lumiracoxib, and etoricoxib.

The active pharmaceutical ingredients in VIOXX® (Merck & Co., Inc.), CELEBREX® (Pfizer, Inc.), BEXTRA® (Pfizer, Inc.), ARCOXIA® (Merck & Co., Inc.) and PREXIGE® (Novartis, Inc.) are rofecoxib, celecoxib, valdecoxib, etoricoxib and lumiracoxib, respectively. Such COX-2 inhibitors have been approved for the treatment of, for example, rheumatoid arthritis, osteoarthritis, and acute pain management in adults.

The present invention provides methods and compositions which decrease or eliminate the risk of adverse cardiovascular and/or renal effects following administration of one or more COX-2 inhibitors (e.g., rofecoxib).

### SUMMARY OF THE INVENTION

In a first aspect, the present invention includes a pharmaceutical composition comprising a COX-2 inhibitor and a diuretic. The compositions and combinations of the present invention can be used to treat or prevent, for example, osteoarthritis, rheumatoid arthritis, acute pain, primary dysmenorrhea, migraine, or colorectal polyps.

In a first embodiment, a pharmaceutical composition comprising rofecoxib and a diuretic is provided. In a specific embodiment, said diuretic is hydrochlorothiazide or chlorthalidone.

In another embodiment, a method of treating inflammation is provided. In another embodiment, a method of treating osteoarthritis is provided. In another embodiment, a method of treating rheumatoid arthritis is provided. In another embodiment, a method of treating pain is provided. In another embodiment, a method of treating dysmenorrhea is provided. In another embodiment, a method of treating migraine is provided. In another embodiment, a method of treating colorectal polyps is provided. The methods comprise administering to a patient an effective amount of a COX-2 inhibitor and a diuretic.

In another embodiment, a method of treating or preventing one or more of inflammation, osteoarthritis, rheumatoid arthritis, pain, dysmenorrhea, migraine, and colorectal polyps is provided, wherein a COX-2 inhibitor is administered in combination with a diuretic. In a specific embodiment, said diuretic is hydrochlorothiazide or chlorthalidone.

In another embodiment, the COX-2 inhibitor and the diuretic can be combined into one dosage unit. In another embodiment, the COX-2 inhibitor and the diuretic can be administered concomitantly as separate dosage forms.

### DETAILED DESCRIPTION OF THE INVENTION

In a first aspect, the present invention includes a pharmaceutical composition comprising a COX-2 inhibitor and a diuretic. Such a composition can be used to treat a patient suffering from, for example, osteoarthritis, rheumatoid arthritis, juvenile rheumatoid arthritis, pain, primary dysmenorrhea, migraine, or colorectal polyps.

The combination of a COX-2 inhibitor and a diuretic for treatment or prevention of such a condition can minimize or eliminate the adverse cardiovascular and/or renal effects, for example, sodium retention, water retention, edema, increased blood pressure, stroke, myocardial infarction, congestive heart failure, or renal dysfunction, associated with one or more COX-2 inhibitors when administered without the incorporation of an appropriate diuretic.

The terms “selective COX-2 inhibitor” and “COX-2 inhibitor” are used interchangeably throughout the disclosure and describe active ingredients that selectively inhibit cyclooxygenase-II enzymes more efficiently than cyclooxygenase-I enzymes.

“COX-2 inhibitors” can be described by the following formula (I) according to the present invention,

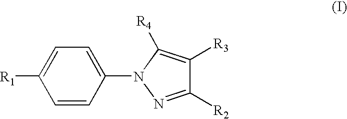

wherein:

R₁ is sulfamyl;

R₂ is haloalkyl;

R₃ is selected from hydrido and alkyl; and

R₄ is selected from aryl, cycloalkyl and cycloalkenyl; wherein R₄ is optionally substituted at a substitutable position with one or more radicals selected from halo, alkylthio, alkylsulfinyl, alkyl, alkylsulfonyl, cyano, carboxyl, alkoxycarbonyl, amido, N-monoalkylamido, N-monoarylamido, N,N-dialkylamido, N-alkyl-N-arylamido, haloalkyl, hydroxyl, alkoxy, hydroxyalkyl, haloalkoxy, sulfamyl, N-alkylsulfamyl, amino, N-alkylamino, N,N-dialkylamino, heterocyclic, nitro and acylamino; or a prodrug thereof or a pharmaceutically acceptable salt, hydrate, or solvate thereof or of said prodrug.

“COX-2 inhibitors” can also be described by the following formula (II) according to the present invention,

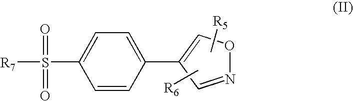

wherein:

R₅ is selected from alkyl, carboxyalkyl, alkoxycarbonyl, aminocarbonyl, aminocarbonylalkyl, alkoxycarbonylalkyl, carboxyl, alkoxy, haloalkoxy, aralkoxy, cycloalkylalkoxy, alkylthio, aralkylthio, cycloalkylalkylthio, alkoxyalkyl, aralkoxyalkyl, alkylthioalkyl, aralkylthioalkyl, alkylaminoalkyl, aryloxyalkyl, arylthioalkyl, hydroxyl, amino, hydroxyalkyl, haloalkyl, cycloalkyl, cycloalkylalkyl, aralkyl, halo, alkylamino, aralkylamino, N-alkyl-N-aralkylamino, N-alkyl-N-cycloalkylalkylamino, arylcarbonyloxyalkyl, arylcarbonylthio, alkoxycarbonyloxyalkyl, alkylaminocarbonyloxyalkyl, alkoxycarbonylthioalkyl and alkylaminocarbonylthioalkyl;

R₆ is selected from cycloalkyl, cycloalkenyl and aryl; wherein R₆ is optionally substituted at a substitutable position with one or more radicals independently selected from alkyl, cyano, carboxyl, alkoxycarbonyl, haloalkyl, hydroxyl, hydroxyalkyl, haloalkoxy, amino, alkylamino, arylamino, aminoalkyl, nitro, alkoxyalkyl, alkylsulfinyl, alkylsulfonyl, aminosulfonyl, halo, alkoxy and alkylthio; and

R₇ is selected from lower alkyl, hydroxyl and amino; or a prodrug thereof or a pharmaceutically acceptable salt, hydrate, or solvate thereof or of said prodrug.

“COX-2 inhibitors” can also be described by the following formula (III) according to the present invention,

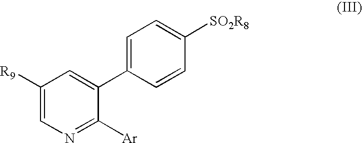

wherein:

R₈ is selected from the group consisting of: CH₃, NH₂, NHC(O)CF₃, and NHCH₃;

Ar is a mono-, di-, or trisubstituted phenyl or pyridinyl (or the N-oxide thereof), wherein the substituents are selected from the group consisting of: hydrogen, halo, C₁₋₆alkoxy, C₁₋₆alkylthio, CN, C₁₋₆alkyl, C₁₋₆fluoroalkyl, N₃, —CO₂R₁₀, hydroxy, —C(R₁₁)(R₁₂)—OH, —C₁₋₆alkyl-CO₂—R₁₃, and C₁₋₆fluoroalkoxy;

R₉ is selected from the group consisting of: halo, C₁₋₆alkoxy, C₁₋₆alkylthio, CN, C₁₋₆alkyl, C₁₋₆fluoroalkyl, N₃, —CO₂R₁₄, hydroxy, —C(R₁₅)(R₁₆)—OH, —C₁₋₆alkyl-CO₂—R₁₇, C₁₋₆fluoroalkoxy, NO₂, NR₁₈R₁₉, and NHCOR₂₀;

R₁₀, R₁₁, R₁₂, R₁₃, R₁₄, R₁₅, R₁₆, R₁₇, R₁₈, R₁₉, and R₂₀, are each independently selected from the group consisting of: hydrogen and C₁₋₆alkyl, or R₁₁ and R₁₂, R₁₅ and R₁₆ or R₁₈ and R₁₉ together with the atom to which they are attached form a saturated monocyclic ring of 3, 4, 5, 6 or 7 atoms.

“COX-2 inhibitors” can also be described by the following formula (IV) according to the present invention,

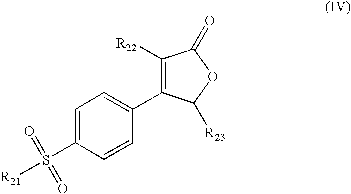

wherein:

R₂₁ is selected from the group consisting of: CH₃, NH₂, NHC(O)CF₃, and NHCH₃;

R₂₂ is selected from the group consisting of: cycloalkyl, cycloalkenyl and aryl; wherein R₂₂ is optionally substituted at a substitutable position with one or more radicals independently selected from the group consisting of: alkyl, cyano, carboxyl, alkoxycarbonyl, haloalkyl, hydroxyl, hydroxyalkyl, haloalkoxy, amino, alkylamino, arylamino, aminoalkyl, nitro, alkoxyalkyl, alkylsulfinyl, alkylsulfonyl, aminosulfonyl, halo, alkoxy and alkylthio; and

R₂₃ is selected from the group consisting of: hydrogen, halo, C₁₋₆alkoxy, C₁₋₆alkylthio, CN, C₁₋₆alkyl, C₁₋₆fluoroalkyl, N₃, —CO₂R₁₀, hydroxy, —C(R₁₁)(R₁₂)—OH, —C₁₋₆alkyl-CO₂—R₁₃, and C₁₋₆fluoroalkoxy.

“COX-2 inhibitors” include, but are not limited to, rofecoxib, celecoxib, valdecoxib, etoricoxib, parecoxib, and deracoxib. Celecoxib, rofecoxib, etoricoxib, lumiracoxib, and valdecoxib are preferred COX-2 inhibitors.

“Diuretics” or “diuretic agents” include, but are not limited to, thiazide and related sulfonamide diuretics bendroflumethiazide, benzthiazide, dyazide, chlorothiazide, chlorthalidone, cyclothiazide, hydrochlorothiazide, hydroflumethiazide, indapamide, methylclothiazide, metolazone, polythiazide, quinethazone and thrichlormethiazide. Other diuretics include the Loop diuretics, such as bumetanide; ethacrynic acid, ethacrynate sodium, and furosemide. The above cited diuretics are known in the art and can be administered in the fashion and at the concentrations known in the art.

“Inflammation” is defined as a response of body tissue to injury or irritation, and can result in an abnormal enlargement of tissue, or a portion thereof. Inflammation can include many conditions such as, but not limited to, asthma, multiple sclerosis, and rheumatoid arthritis.

In a first embodiment, a pharmaceutical composition comprising rofecoxib and a diuretic is provided. In a specific embodiment, said diuretic is hydrochlorothiazide or chlorthalidone.

In another embodiment, a pharmaceutical composition comprising celecoxib and a diuretic is provided. In a specific embodiment, said diuretic is hydrochlorothiazide or chlorthalidone.

In another embodiment, a pharmaceutical composition comprising valdecoxib and a diuretic is provided. In a specific embodiment, said diuretic is hydrochlorothiazide or chlorthalidone.

In another embodiment, a pharmaceutical composition comprising etoricoxib and a diuretic is provided. In a specific embodiment, said diuretic is hydrochlorothiazide or chlorthalidone.

In another embodiment, a pharmaceutical composition comprising lumiracoxib and a diuretic is provided. In a specific embodiment, said diuretic is hydrochlorothiazide or chlorthalidone.

In another embodiment, the present invention provides a method of minimizing or eliminating adverse cardiovascular and/or renal effects associated with the administration of one or more COX-2 inhibitors. In one embodiment, said method comprises administering to a patient a COX-2 inhibitor and a diuretic.

In another embodiment, a method of treating inflammation is provided. The method comprises administering to a patient an effective amount of a COX-2 inhibitor and a diuretic.

In a more particular embodiment of the invention, a method of treating inflammation in a patient in need of such treatment is provided, comprising administering to said patient a COX-2 inhibitor and a diuretic in an amount that is effective to treat inflammation.

In a specific embodiment, a method of treating inflammation in a patient in need of such treatment is provided, comprising administering to said patient a COX-2 inhibitor and a diuretic in an amount that is effective to treat inflammation, wherein said COX-2 inhibitor is selected from the group consisting of: rofecoxib, celecoxib, valdecoxib, etoricoxib, lumiracoxib, and parecoxib, and said diuretic is selected from the group consisting of: thiazide, bendroflumethiazide, benzthiazide, dyazide, chlorothiazide, chlorthalidone, cyclothiazide, hydrochlorothiazide, hydroflumethiazide, indapamide, methylclothiazide, metolazone, polythiazide, quinethazone, thrichlormethiazide, bumetanide, ethacrynic acid, ethacrynate salt (e.g., sodium salt), and furosemide.

In another embodiment, a method of treating inflammation is provided, wherein rofecoxib is administered in combination with a diuretic. In a specific embodiment, said diuretic is hydrochlorothiazide.

In another embodiment, a method of treating osteoarthritis is provided. The method comprises administering to a patient an effective amount of a COX-2 inhibitor and a diuretic.

In a more particular embodiment of the invention, a method of treating osteoarthritis in a patient in need of such treatment is provided, comprising administering to said patient a COX-2 inhibitor and a diuretic in an amount that is effective to treat osteoarthritis.

In a specific embodiment, a method of treating osteoarthritis in a patient in need of such treatment is provided, comprising administering to said patient a COX-2 inhibitor and a diuretic in an amount that is effective to treat osteoarthritis, wherein said COX-2 inhibitor is selected from the group consisting of: rofecoxib, celecoxib, valdecoxib, etoricoxib, lumiracoxib, and parecoxib, and said diuretic is selected from the group consisting of: thiazide, bendroflumethiazide, benzthiazide, dyazide, chlorothiazide, chlorthalidone, cyclothiazide, hydrochlorothiazide, hydroflumethiazide, indapamide, methylclothiazide, metolazone, polythiazide, quinethazone, thrichlormethiazide, bumetanide, ethacrynic acid, ethacrynate salt (e.g., sodium salt), and furosemide.

In another embodiment, a method of treating osteoarthritis is provided, wherein rofecoxib is administered in combination with a diuretic. In a specific embodiment, said diuretic is hydrochlorothiazide.

In another embodiment, the COX-2 inhibitor and the diuretic can be combined into one dosage unit. In another embodiment, the COX-2 inhibitor and the diuretic can be taken concomitantly as separate dosage forms. In a specific embodiment, a dosage unit comprises a tablet including a first layer or portion containing the COX-2 inhibitor, and a second layer or portion containing at least one diuretic. Each of said layers or portions can be substantially homogenous, i.e., the COX-2 inhibitor/diuretic ingredient in each of the layers can be substantially uniformly distributed throughout the respective layers. Said layers or portions can be formed directly adjacent each other, without any intervening barrier layer. Physical separation of the COX-2 inhibitor/diuretic ingredients into two different portions or layers, without a separating barrier, may be sufficient to prevent any degradation of the COX-2 inhibitor due to contact with the diuretic or any degradation of the diuretic due to contact with the COX-2 inhibitor. In another embodiment, the physical separation of the layers or portions is accomplished with a separating barrier. In another embodiment, the COX-2 inhibitor and the diuretic are combined or mixed into one layer or portion. In such an embodiment, the layer or portion can be substantially homogeneous, for example, a homogeneous tablet or capsule.

In another embodiment, a method of preventing any one or more of the conditions discussed herein, including, but not limited to, inflammation, osteoarthritis, rheumatoid arthritis, pain, dysmenorrhea, migraine, and colorectal polyps is provided. Said method of preventing comprises administering a pharmaceutical composition or formulation of the present invention to a patient in need thereof.

Pharmaceutical compositions and formulations of the present invention would be useful to treat arthritis, including but not limited to rheumatoid arthritis, spondyloarthropathies, gouty arthritis, osteoarthritis, systemic lupus erythematosus, juvenile arthritis, and pain due to arthritis.

The pharmaceutical compositions and formulations of COX-2 inhibitors including celecoxib and valdecoxib described in International Applications WO04/000284, WO04/061433, and WO04/026235 can likewise be combined with an appropriate diuretic in order to minimize or eliminate adverse cardiovascular and/or renal effects. WO04/000284, WO04/061433, and WO04/026235 are herein incorporated by reference in their entireties.

In another embodiment, a pharmaceutical composition or formulation comprises a therapeutically effective amount of a COX-2 inhibitor and an amount of a diuretic sufficient to maintain the patient's blood pressure within about 5 percent or less of their average blood pressure prior to administration of said COX-2 inhibitor. In another embodiment, a pharmaceutical composition or formulation comprises a therapeutically effective amount of a COX-2 inhibitor and an amount of a diuretic sufficient to maintain the patient's blood pressure within about 4 percent or less of their average blood pressure prior to administration of said COX-2 inhibitor. In another embodiment, a pharmaceutical composition or formulation comprises a therapeutically effective amount of a COX-2 inhibitor and an amount of a diuretic sufficient to maintain the patient's blood pressure within about 3 percent or less of their average blood pressure prior to administration of said COX-2 inhibitor.

In another embodiment, a pharmaceutical composition or formulation comprises a therapeutically effective amount of a COX-2 inhibitor and an amount of a diuretic sufficient to maintain the patient's blood pressure within about+/−5 mm Hg of their average blood pressure prior to administration of said COX-2 inhibitor.

The “average blood pressure” can be calculated by one or more means known to those of ordinary skill in the art. For example, the calculation can be made by determining the mean arterial pressure (MAP) at each of several time points and calculating an average value.

In another embodiment, the present invention provides a pharmaceutical composition comprising a COX-2 inhibitor and a diuretic, wherein sodium retention is decreased by at least about 10 percent relative to administration of said COX-2 inhibitor without said diuretic. In another embodiment, the present invention provides a pharmaceutical composition comprising a COX-2 inhibitor and a diuretic, wherein sodium retention is decreased by at least about 20 percent relative to administration of said COX-2 inhibitor without said diuretic. In another embodiment, the present invention provides a pharmaceutical composition comprising a COX-2 inhibitor and a diuretic, wherein sodium retention is decreased by at least about 30 percent relative to administration of said COX-2 inhibitor without said diuretic. In another embodiment, the present invention provides a pharmaceutical composition comprising a COX-2 inhibitor and a diuretic, wherein sodium retention is decreased by at least about 40 percent relative to administration of said COX-2 inhibitor without said diuretic. In another embodiment, the present invention provides a pharmaceutical composition comprising a COX-2 inhibitor and a diuretic, wherein sodium retention is decreased by at least about 50 percent relative to administration of said COX-2 inhibitor without said diuretic. In another embodiment, the present invention provides a pharmaceutical composition comprising a COX-2 inhibitor and a diuretic, wherein sodium retention is decreased by at least about 60 percent relative to administration of said COX-2 inhibitor without said diuretic. In another embodiment, the present invention provides a pharmaceutical composition comprising a COX-2 inhibitor and a diuretic, wherein sodium retention is decreased by at least about 70 percent relative to administration of said COX-2 inhibitor without said diuretic. In another embodiment, the present invention provides a pharmaceutical composition comprising a COX-2 inhibitor and a diuretic, wherein sodium retention is decreased by at least about 80 percent relative to administration of said COX-2 inhibitor without said diuretic. In another embodiment, the present invention provides a pharmaceutical composition comprising a COX-2 inhibitor and a diuretic, wherein sodium retention is decreased by at least about 90 percent relative to administration of said COX-2 inhibitor without said diuretic. In another embodiment, the present invention provides a pharmaceutical composition comprising a COX-2 inhibitor and a diuretic, wherein sodium retention is decreased by about 100 percent relative to administration of said COX-2 inhibitor without said diuretic.

In another embodiment, the present invention provides a pharmaceutical composition comprising a COX-2 inhibitor and a diuretic, wherein water retention is decreased by at least about 10 percent relative to administration of said COX-2 inhibitor without said diuretic. In another embodiment, the present invention provides a pharmaceutical composition comprising a COX-2 inhibitor and a diuretic, wherein water retention is decreased by at least about 20 percent relative to administration of said COX-2 inhibitor without said diuretic. In another embodiment, the present invention provides a pharmaceutical composition comprising a COX-2 inhibitor and a diuretic, wherein water retention is decreased by at least about 30 percent relative to administration of said COX-2 inhibitor without said diuretic. In another embodiment, the present invention provides a pharmaceutical composition comprising a COX-2 inhibitor and a diuretic, wherein water retention is decreased by at least about 40 percent relative to administration of said COX-2 inhibitor without said diuretic. In another embodiment, the present invention provides a pharmaceutical composition comprising a COX-2 inhibitor and a diuretic, wherein water retention is decreased by at least about 50 percent relative to administration of said COX-2 inhibitor without said diuretic. In another embodiment, the present invention provides a pharmaceutical composition comprising a COX-2 inhibitor and a diuretic, wherein water retention is decreased by at least about 60 percent relative to administration of said COX-2 inhibitor without said diuretic. In another embodiment, the present invention provides a pharmaceutical composition comprising a COX-2 inhibitor and a diuretic, wherein water retention is decreased by at least about 70 percent relative to administration of said COX-2 inhibitor without said diuretic. In another embodiment, the present invention provides a pharmaceutical composition comprising a COX-2 inhibitor and a diuretic, wherein water retention is decreased by at least about 80 percent relative to administration of said COX-2 inhibitor without said diuretic. In another embodiment, the present invention provides a pharmaceutical composition comprising a COX-2 inhibitor and a diuretic, wherein water retention is decreased by at least about 90 percent relative to administration of said COX-2 inhibitor without said diuretic. In another embodiment, the present invention provides a pharmaceutical composition comprising a COX-2 inhibitor and a diuretic, wherein water retention is decreased by about 100 percent relative to administration of said COX-2 inhibitor without said diuretic.

In another embodiment, the present invention provides a pharmaceutical composition comprising a COX-2 inhibitor and a diuretic, wherein upon administration the peak plasma concentrations of the COX-2 inhibitor and of the diuretic occur within about four hours of each other. In another embodiment, the present invention provides a pharmaceutical composition comprising a COX-2 inhibitor and a diuretic, wherein upon administration the peak plasma concentrations of the COX-2 inhibitor and of the diuretic occur within about two hours of each other. In another embodiment, the present invention provides a pharmaceutical composition comprising a COX-2 inhibitor and a diuretic, wherein upon administration the peak plasma concentrations of the COX-2 inhibitor and of the diuretic occur within about one hour of each other. In another embodiment, the present invention provides a pharmaceutical composition comprising a COX-2 inhibitor and a diuretic, wherein upon administration the peak plasma concentrations of the COX-2 inhibitor and of the diuretic occur within about 30 minutes of each other.

Excipients employed in pharmaceutical compositions of the present invention can be solids, semi-solids, liquids or combinations thereof. Preferably, excipients are solids. Compositions of the invention containing excipients can be prepared by any known technique of pharmacy that comprises admixing an excipient with a drug or therapeutic agent. A pharmaceutical composition of the invention contains a desired amount of COX-2 inhibitor and a diuretic per dose unit and, if intended for oral administration, can be in the form, for example, of a tablet, a caplet, a pill, a hard or soft capsule, a lozenge, a cachet, a dispensable powder, granules, a suspension, an elixir, a dispersion, a liquid, or any other form reasonably adapted for such administration. Presently preferred are oral dosage forms that are discrete dose units each containing a predetermined amount of the COX-2 inhibitor and the diuretic, such as tablets or capsules.

Pharmaceutical compositions of the invention optionally comprise, but are not limited to, one or more pharmaceutically acceptable carriers, diluents, disintegrants, binding agents, adhesives, wetting agents, lubricants, anti-adherents, glidants, antioxidants, surfactants, or effervescent agents as excipients. Specific excipients can be found in various literature references, such as the Handbook of Pharmaceutical Excipients, 4^{th} edition, by Rowe et al., or are known to those of ordinary skill in the art.

Solid dosage forms of the invention can be prepared by any suitable process, and are not limited to processes described herein. An illustrative process comprises (i) a step of blending a COX-2 inhibitor and a diuretic with one or more excipients to form a blend, and (ii) a step of tableting or encapsulating the blend to form tablets or capsules, respectively.

Excipients for tablet compositions of the invention are preferably selected to provide a disintegration time of less than about 30 minutes, preferably about 25 minutes or less, more preferably about 20 minutes or less, and still more preferably about 15 minutes or less, in a standard disintegration assay.

COX-2 inhibitor dosage forms of the invention preferably comprise a daily dosage amount of about 1 mg to about 1000 mg, more preferably about 1 mg to about 100 mg, about 100 mg to about 150 mg, 150 mg to about 200 mg, 200 mg to about 250 mg, 250 mg to about 300 mg, 300 mg to about 350 mg, 350 mg to about 400 mg, 400 mg to about 450 mg 450 mg to about 500 mg, 500 mg to about 550 mg, 550 mg to about 600 mg, 600 mg to about 700 mg, or 700 mg to about 800 mg.

Pharmaceutical compositions of the invention comprise one or more orally deliverable dose units. Each dose unit comprises a COX-2 inhibitor and a diuretic in a therapeutically effective amount. The term “dose unit” herein means a portion of a pharmaceutical composition that contains an amount of a therapeutic or prophylactic agent, suitable for a single oral administration to provide a therapeutic effect. Typically one dose unit, or a small plurality (up to about 4) of dose units, in a single administration provides a dose comprising a sufficient amount of the agent to result in the desired effect. Administration of such doses can be repeated as required, typically at a dosage frequency of 1, 2, 3, or 4 times per day.

It will be understood that a therapeutically effective amount of a COX-2 inhibitor and a diuretic for a subject is dependent inter alia on the body weight of the subject. A “subject” to which a COX-2 inhibitor and a diuretic or a pharmaceutical composition thereof can be administered includes a human subject of either sex and of any age. When the subject is a child, for example, an amount of a COX-2 inhibitor relatively low in the preferred range of about 1 mg to about 800 mg is likely to provide blood serum concentrations consistent with therapeutic effectiveness. Where the subject is an adult human, achievement of such blood serum concentrations of a COX-2 inhibitor is likely to require dose units containing a relatively greater amount. Where animals (e.g., dogs, horses, etc.) appropriately respond to the administration of both a COX-2 inhibitor and a diuretic, the methods and pharmaceutical compositions of the present invention can also be applied to same.

Typical dose units of currently available COX-2 inhibitors in a pharmaceutical composition of the invention contain about 10, 15, 20, 25, 37.5, 50, 75, 100, 125, 150, 175, 200, 250, 300, 350, or 400 mg of a COX-2 inhibitor. For an adult human, a therapeutically effective amount of a COX-2 inhibitor per dose unit in a composition of the present invention is typically about 10 mg to about 400 mg. Other doses that are not in current use may become preferred, if the bioavailability is changed with a novel formulation or if new, more potent COX-2 inhibitors are discovered.

A dose unit containing a particular amount of a COX-2 inhibitor can be selected to accommodate any desired frequency of administration used to achieve a desired daily dosage. The daily dosage and frequency of administration, and therefore the selection of appropriate dose unit, depends on a variety of factors, including the age, weight, sex and medical condition of the subject, and the nature and severity of the condition or disorder, and thus may vary widely.

For pain management, pharmaceutical compositions of the present invention can be used to provide a daily dosage of a COX-2 inhibitor of about 10 mg to about 1000 mg, preferably about 10 mg to about 600 mg, more preferably about 10 mg to about 500 mg, and still more preferably about 10 mg to about 400 mg. For example, a daily dose of celecoxib of about 0.7 to about 13 mg/kg body weight, preferably about 1.3 to about 8 mg/kg body weight, more preferably about 2 to about 6.7 mg/kg body weight, and still more preferably about 2.3 to about 5.3 mg/kg body weight, for example about 2.7 mg/kg body weight, is generally appropriate when administered in a pharmaceutical composition of the invention. The daily dose can be administered in one to about four doses per day. Administration at a rate of one 50 mg dose unit four times a day, one 100 mg dose unit or two 50 mg dose units twice a day, or one 200 mg dose unit, two 100 mg dose units or four 50 mg dose units once a day is preferred.

The term “oral administration” herein includes any form of delivery of a therapeutic agent or a composition thereof to a subject wherein the agent or composition is placed in the mouth of the subject, whether or not the agent or composition is immediately swallowed, although each are embodiments of the invention. Thus, “oral administration” includes buccal and sublingual administration. Absorption of the agent can occur in any part or parts of the gastrointestinal tract including the mouth, esophagus, stomach, duodenum, ileum and colon. The term “orally deliverable” herein means suitable for oral administration.

An important aspect of the administration of drugs in conventional forms is the fluctuation between high and low blood serum concentration of the drug in the period between the administration of two successive doses. In fact, if the drug is too rapidly absorbed, excessive plasma levels may be attained, leading to undesirable and even toxic side effects. On the other hand, drugs possessing a short half-life are eliminated too rapidly and require therefore frequent administrations. In both cases the patient must be careful because particular attention and constancy in the administration is required during therapy and such conditions cannot always be easily obtained. Many efforts have been made to formulate pharmaceutical preparations able to protract in time the activity of the drug in the body at optimum plasma levels, reducing the number of administrations and thus improving the response of the patient to the treatment.

The present invention is further directed to a therapeutic method of treating a condition or disorder where treatment with a COX-2 inhibitory drug is indicated, the method comprising oral administration of a pharmaceutical composition of the invention to a subject in need thereof. The dosage regimen to prevent, give relief from, or ameliorate the condition or disorder preferably corresponds to once-a-day or twice-a-day treatment, but can be modified in accordance with a variety of factors. These include the type, age, weight, sex, diet and medical condition of the subject and the nature and severity of the disorder. Thus, the dosage regimen actually employed can vary widely and can therefore deviate from the preferred dosage regimens set forth above. The present pharmaceutical compositions can be used in combination with other therapies or therapeutic agents, including but not limited to, therapies with narcotic analgesics, non-NSAID analgesics (e.g., acetaminophen), GABA active agents, and sodium channel blockers, among others.

These pharmaceutical compositions can be used to treat subjects having adenomatous polyps, including those with familial adenomatous polyposis (FAP). Additionally, pharmaceutical compositions of the present invention can be used to prevent polyps from forming in subjects at risk of FAP.

Preferred uses for pharmaceutical compositions of the invention are for treatment of rheumatoid arthritis and osteoarthritis, for pain management generally (particularly post-oral surgery pain, post-general surgery pain, post-orthopedic surgery pain, and acute flares of osteoarthritis), and for colon cancer chemoprevention. A particular preferred use is for rapid pain management, such as when a COX-2 inhibitor or a pharmaceutical composition thereof is effective in treating pain within about 30 minutes or less.

The active agents of this invention are preferably given orally, as needed.

In one specific embodiment of the present invention rofecoxib is administered to the recipient at a daily dose of from about 12.5 mg to about 50 mg, preferably from about 12.5 mg to about 25 mg, in conjunction with hydrochlorothiazide administration at a daily concentration of from about 10 mg to about 100 mg, more preferably from about 12.5 mg to about 50 mg. The doses of this combination regimen are preferably administered at the same time(s) per day and may be administered once per day or divided into two or more doses.

Preferably, the formulations of this invention are enclosed in a solid dosage form after manufacture, such as a tablet. The formulations of this invention may also be created as a liquid or semi-liquid formulation and introduced into a capsule. Similarly, using an acceptable range of components and/or temperatures, the formulation may be made as a gel or solid prior to encapsulation.

### EXEMPLIFICATION

### Example 1

### A Study of Decreasing the Risk of Adverse Cardiovascular Effects in Humans

In order to study the adverse cardiovascular effects of VIOXX® (or other COX-2 inhibitors) in humans, a randomized, double-blind study in elderly volunteer patients can be completed. The study can consist of a number of patients, for example, 25 to 30 patients, and comprising four 2-week periods with a 1-week washout between each period. The four periods should consist of the administration of: a placebo, 25 mg VIOXX®, 25 mg VIOXX® and 12.5 mg hydrochlorothiazide, and 25 mg VIOXX® and 25 mg hydrochlorothiazide. During each period, urine samples can be collected every 24 hours and tested for sodium, potassium, and creatinine content. Patients' weight and blood pressure can also be monitored before, during, and after such a study. Optionally, an outcome study (quantifying significant cardiovascular and renal side effects, including, but not limited to, edema, hypertension, congestive heart failure, heart attack, and stroke) can also be completed to further study the effects of administration of hydrochlorothiazide with VIOXX®.

What is claimed is:

1. A pharmaceutical composition comprising a COX-2 inhibitor and a diuretic.

2. The pharmaceutical composition of claim 1, wherein:
(a) said COX-2 inhibitor is rofecoxib;
(b) said COX-2 inhibitor is celecoxib;
(c) said COX-2 inhibitor is valdecoxib;
(d) said COX-2 inhibitor is etoricoxib;
(e) said COX-2 inhibitor is lumiracoxib;
(f) said diuretic is hydrochlorothiazide;
(g) said COX-2 inhibitor is rofecoxib and said diuretic is hydrochlorothiazide;
(h) said COX-2 inhibitor is celecoxib and said diuretic is hydrochlorothiazide;
(i) said COX-2 inhibitor is valdecoxib and said diuretic is hydrochlorothiazide;
(j) said COX-2 inhibitor is selected from the group consisting of: rofecoxib, celecoxib, valdecoxib, lumiracoxib, and etoricoxib; or
(k) said diuretic is selected from the group consisting of: thiazide, bendroflumethiazide, benzthiazide, dyazide, chlorothiazide, chlorthalidone, cyclothiazide, hydrochlorothiazide, hydroflumethiazide, indapamide, methylclothiazide, metolazone, polythiazide, quinethazone, thrichlormethiazide, bumetanide, ethacrynic acid, ethacrynate sodium, and furosemide.

3. The pharmaceutical composition of claim 1, further comprising an excipient.

4. A method of treating inflammation or osteoarthritis, comprising administering an effective amount of a COX-2 inhibitor and a diuretic to a patient in need thereof.

5. The method of claim 4, wherein:
(a) said COX-2 inhibitor is rofecoxib;
(b) said COX-2 inhibitor is celecoxib;
(c) said COX-2 inhibitor is valdecoxib;
(d) said COX-2 inhibitor is etoricoxib;
(e) said COX-2 inhibitor is lumiracoxib;
(f) said diuretic is hydrochlorothiazide; or
(g) said COX-2 inhibitor is rofecoxib and said diuretic is hydrochlorothiazide;
(h) said COX-2 inhibitor is celecoxib and said diuretic is hydrochlorothiazide;
(i) said COX-2 inhibitor is valdecoxib and said diuretic is hydrochlorothiazide;
(j) said COX-2 inhibitor and said diuretic are administered in a single dosage form; or
(k) said COX-2 inhibitor and said diuretic are administered in separate dosage forms.

6. A method of decreasing or eliminating the risk to a patient being treated with a COX-2 inhibitor of suffering an adverse cardiovascular or renal effect, comprising administering an effective amount of a COX-2 inhibitor and a diuretic.

7. The method of claim 6, wherein:
(a) said COX-2 inhibitor is rofecoxib;
(b) said COX-2 inhibitor is celecoxib;
(c) said COX-2 inhibitor is valdecoxib;
(d) said COX-2 inhibitor is etoricoxib;
(e) said COX-2 inhibitor is lumiracoxib;
(f) said diuretic is hydrochlorothiazide; or
(g) said COX-2 inhibitor is rofecoxib and said diuretic is hydrochlorothiazide;
(h) said COX-2 inhibitor is celecoxib and said diuretic is hydrochlorothiazide;
(i) said COX-2 inhibitor is valdecoxib and said diuretic is hydrochlorothiazide;
(j) said COX-2 inhibitor and said diuretic are administered in a single dosage form; or
(k) said COX-2 inhibitor and said diuretic are administered in separate dosage forms.
